(19) United States
(12) Patent Application Publication
(10) Pub. No.: US 2003/0044989 A1
(43) Pub. Date: Mar. 6, 2003

(54) Apparatus and method for testing a beverage for a clandestine illicit substance

(76) Inventors: Francisco Javier Guerra, Plantation, FL (US); Brian Robert Glover, Belle Terre, NY (US); James S.T. McCafferty, Fort Lauderdale, FL (US)

Correspondence Address: David W. Barman, 1101 NE 176 Terrace, North Miami Beach, FL, 33162, US

(21) Appl. No.: 09/945,908

(22) Filed: Sep. 4, 2001

(51) Int. Cl. G01N 33/14

(52) U.S. Cl. 436/24; 436/20; 436/169; 422/56; 422/57

(57) ABSTRACT

An apparatus and method for detecting the clandestine placement of an illicit chemical present in a beverage is disclosed and described.

### FIELD OF THE INVENTION

An apparatus and method for detecting the clandestine placement of an illicit chemical present in a beverage is disclosed and described. More particularly, an apparatus and method by which an individual may safely and rapidly perform a qualitative assay to determine if a beverage has been subject to unwanted addition of extraneous chemical entities.

### BACKGROUND OF THE INVENTION

There is growing concern over a relatively new crime, date rape. The perpetrators of this heinous act have resorted to approaching their victims at parties, bars and social gatherings, and succeeded in the clandestine placement of various chemical entities into the beverages of their victims. The victim, unaware that tampering has taken place, consumes the beverage and is rendered into a state such that defense against their attacker is a virtual impossibility. There are many such chemical entities at the disposal of the rapist. They have been collectively termed date rape drugs. These include, but are not limited to: Flunitrazepam (also known as Rohypnol), Ketamine, and Gamma hyroxybutyrate (GHB). These and many others can greatly affect the victims' consciousness and ability to defend in the event of an attack. Chemical testing for these substances is very well documented. However, what is not available is an apparatus and means for individuals to test their beverages, in their social setting, if they suspect tampering has taken place.

It is the object of the invention to provide an apparatus and method for detecting a clandestine chemical entity in a beverage that is easy to use, reliable, safe, and inexpensive to mass-produce.

### DETAILED DESCRIPTION OF THE PREFERRED EMBODIMENTS

The apparatus is intended for the rapid, easy and reliable testing of date rape drugs. Date rape drugs are defined as those substances, which are used by an assailant to render the victim into a state of consciousness, which may be conscious, semi-conscious or unconscious, such that the victim loses the ability of self-defense. These date rape drugs can include but are not limited to: Flunitrazepam, which is commonly known as Rohypnol or “Ruffies,” 4-Hydroxybutanoic acid, also known as gamma hydroxy butyrate (GHB) and Ketamine. The apparatus is composed with one or more solid, chemical colorimetric indicators embedded in the surface of the invention. The apparatus should be of suitable porosity so as to allow the flow of the test solution to reach said colorimetric indicator. The invention can be used in, but are not limited to: a cocktail napkin, beverage coaster, placemat, menu, match book, drink carrier, flyer, coupon, personal test kit or even a business card. The manufacturing of the apparatus is to be in a manner such that the test regions are clearly discernable to the user. The apparatus can even be manufactured in a manner to include an advertisement or a logo. The method of use would comprise the steps of: locating a specific region on the apparatus, removing a drop of beverage, placing the drop within a marked region on the apparatus, observing a colorimetric indication within the region wherein the drop was placed. The removal can be done using a straw, a swizzle stick or even one's finger. Each region would be specific for an individual compound. The invention may contain one or more marked regions in order to test for more than one illicit substance. A qualitative colorimetric result would then instantly be observed. These colorimetric indicator test spots provide for colors that are bright and distinctive. In doing so, the test result would not be confused with being a byproduct of the beverage color.

The testing for illicit substances is well known in the chemical arts. Flunitrazepam, which is commonly known as Rohypnol or “Ruffies” is a member of the class of compounds known as benzodiazopines. Either a reaction with Zimmermann's reagent, or reacting with a platinum/potassium iodide test system can detect this class of compound. 4-Hydroxybutanoic acid, also known as gamma hydroxy butyrate (GHB) is a commonly known anesthetic. It can be identified in a reaction system with bromo cresol purple. Ketamine is another anesthetic for which the current invention can be applied. It can be identified using cobalt thiocyanate.

Another embodiment provides for the test material to be deposited on a solid, non-porous substrate, such as a plate or glass.

These are provided by way of example and are in no means intended to be limiting the scope of the invention.

While the invention has been described in its preferred form or embodiment with some degree of particularity, it is understood that this description has been given only by way of example and that numerous changes in the details of construction, fabrication, and use, including the combination and arrangement of parts, may be made without departing from the spirit and scope of the invention.

We claim:

1. An apparatus for detecting the presence of an illicit substance in a beverage comprising:
a) a manufactured porous substrate
b) one or more calorimetric indicators embedded in or upon said substrate
c) optionally, indication of the placement of said colorimetric indicators.

2. The apparatus of claim 1 where said illicit substance is a date rape drug.

3. The apparatus of claim 1 where said illicit substance is Flunitrazepam.

4. The apparatus of claim 1 where said illicit substance is 4-Hydroxybutanoic acid.

5. The apparatus of claim 1 where said illicit substance is Ketamine.

6. The apparatus of claim 1 where the manufactured porous substrate is made with sufficient porosity to allow the flow of the test indicator solution through said substrate.

7. The apparatus of claim 1 where the manufactured porous substrate is a napkin.

8. The apparatus of claim 1 where the manufactured porous substrate is the paper lining of a beverage coaster, placemat, menu, match book, drink carrier, flyer, coupon, personal test kit or business card.

9. The apparatus of claim 1 where the manufactured porous substrate is a business card.

10. The apparatus of claim 1 where said substrate contains at least one embedded calorimetric indicator.

11. The colorimetric indicator of claim 1, where said indicator is suitable for testing the presence of an illicit substance.

12. The calorimetric indicator of claim 1, where said indicator is Zimmermann's reagent.

13. The colorimetric indicator of claim 1, where said indicator is platinum/potassium iodide.

14. The colorimetric indicator of claim 1, where said indicator is bromo cresol purple.

15. The calorimetric indicator of claim 1, where said indicator is cobalt thiocyanate.

16. A method for testing for the presence of an illicit substance present in a beverage which comprises the steps of:
a) removing a sample of solution from the beverage to be tested,
b) placing said sample on at least one marked region of a testing substrate,
c) observing the qualitative result as a calorimetric change.

17. The method of claim 17 where said sample weighs at least one twentieth of a gram.

18. The method of claim 17 where said marked regions are labeled for specific substances.

19. The method of claim 17 where said colorimetric change rapidly occurs and is easily discernable.
